(19) United States
(12) Patent Application Publication
(10) Pub. No.: US 2022/0202380 A1
(43) Pub. Date: Jun. 30, 2022

(54) METHOD FOR MONITORING PEDIATRIC COLONIC MOTILITY

(71) Applicant: Konsyl Pharmaceuticals, Inc, Easton, MD (US)

(72) Inventor: Frank Gunsallus, III, Trappe, MD (US)

(73) Assignee: Konsyl Pharmaceuticals, Inc, Easton, MD (US)

(21) Appl. No.: 17/601,070

(22) PCT Filed: Apr. 6, 2020
(86) PCT No.: PCT/US2020/026811

§ 371 Date: Oct. 3, 2021

Related U.S. Application Data
(60) Provisional application No. 62/829,226, filed on Apr. 4, 2019.

(51) Int. Cl. A61B 6/12; A61B 90/00; A61K 49/04

(52) U.S. Cl. CPC A61B 6/12; A61B 90/39; A61B 2090/3966; A61K 49/0409; A61K 49/0404

(57) ABSTRACT

Embodiments are directed to a method for monitoring pediatric colonic motility having the steps of administering an ingestible and dissolvable capsule to a pediatric patient, wherein the capsule contains a plurality of radiopaque markers; and performing a radiograph on the pediatric patient to identify the location of at least one of the plurality of radiopaque markers within the pediatric patient's colon.

### CROSS-REFERENCE TO RELATED APPLICATIONS

This patent application claims priority to U.S. provisional patent application 62/829,226 filed on Apr. 4, 2019. The subject matter from U.S. provisional patent application 62/829,226 is incorporated by reference into this application.

### BACKGROUND OF THE INVENTION

Although methods for monitoring adult colonic motility are known, governmental regulations have previously presented limitations on methods directed to monitoring pediatric colonic motility. Because of that, a need remains for methods directed to monitoring pediatric colonic motility.

### BRIEF SUMMARY OF THE INVENTION

Embodiments are directed to a method for monitoring pediatric colonic motility having the steps of administering an ingestible and dissolvable capsule to a pediatric patient, wherein the capsule contains a plurality of radiopaque markers; and performing a radiograph on the pediatric patient to identify the location of at least one of the plurality of radiopaque markers within the pediatric patient's colon.

Embodiments are directed to a method for monitoring pediatric colonic motility having the steps of administering an ingestible and dissolvable capsule to a pediatric patient having an age ranging from 2 to 17 years old, wherein the capsule contains a plurality of radiopaque markers; and performing a radiograph on the pediatric patient to identify the location of at least one of the plurality of radiopaque markers within the pediatric patient's colon; wherein the radiograph is performed less than six days after the pediatric patient ingests the ingestible capsule, wherein the ingestible capsule contains at least 5 radiopaque markers, wherein there are at least three different shapes of radiopaque markers: a first kind of marker having a perimeter shape that is substantially circular and having a first internal configuration; a second kind of marker having a perimeter shape that is substantially circular and having a second internal configuration, and a third kind of marker having a perimeter shape that is substantially circular and having a third internal configuration, and wherein all of the radiopaque markers have a perimeter shape that is substantially circular and a diameter that is less than 0.180 inches.

Embodiments are directed to a method for monitoring pediatric colonic motility having the steps of administering an ingestible and dissolvable capsule to a pediatric patient having an age ranging from 2 to 17 years old, wherein the capsule contains 24 radiopaque markers; and performing a radiograph on the pediatric patient to identify the location of at least one of the plurality of radiopaque markers within the pediatric patient's colon; wherein the radiograph is performed less than six days after the pediatric patient ingests the ingestible capsule, wherein the ingestible capsule contains at least 5 radiopaque markers, wherein there are at least three different shapes of radiopaque markers: a first kind of marker having a perimeter shape that is substantially circular and having a first internal configuration; a second kind of marker having a perimeter shape that is substantially circular and having a second internal configuration, and a third kind of marker having a perimeter shape that is substantially circular and having a third internal configuration, and wherein all of the radiopaque markers have a perimeter shape that is substantially circular and a diameter that is less than 0.180 inches.

### BRIEF DESCRIPTION OF THE SEVERAL VIEWS OF THE DRAWING

FIG. 1 shows a two-dimensional view of three separate embodiments of useful radiopaque markers.

FIG. 2A shows an embodiment of an ingestible and dissolvable capsule filled with a plurality of radiopaque markers.

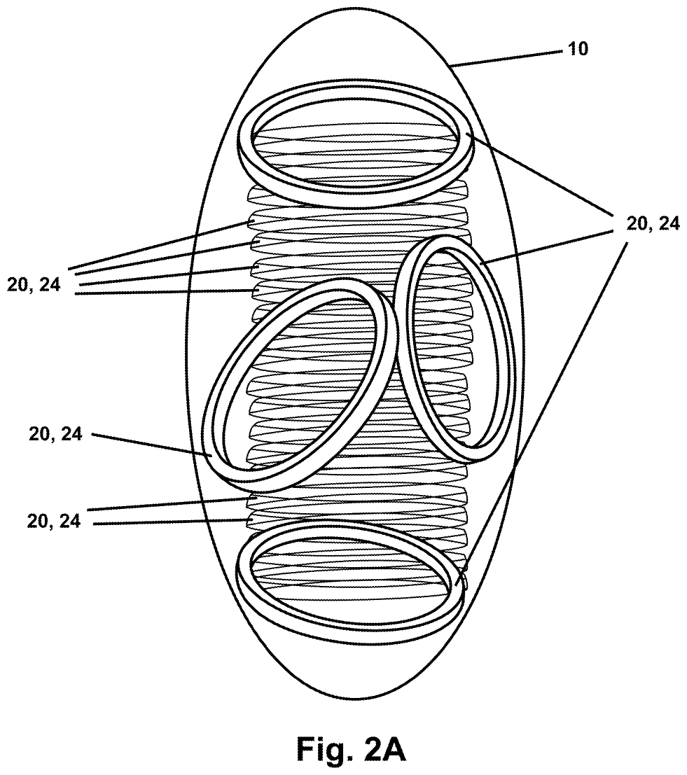

FIG. 2B shows an embodiment of an ingestible and dissolvable capsule filled with a plurality of radiopaque markers.

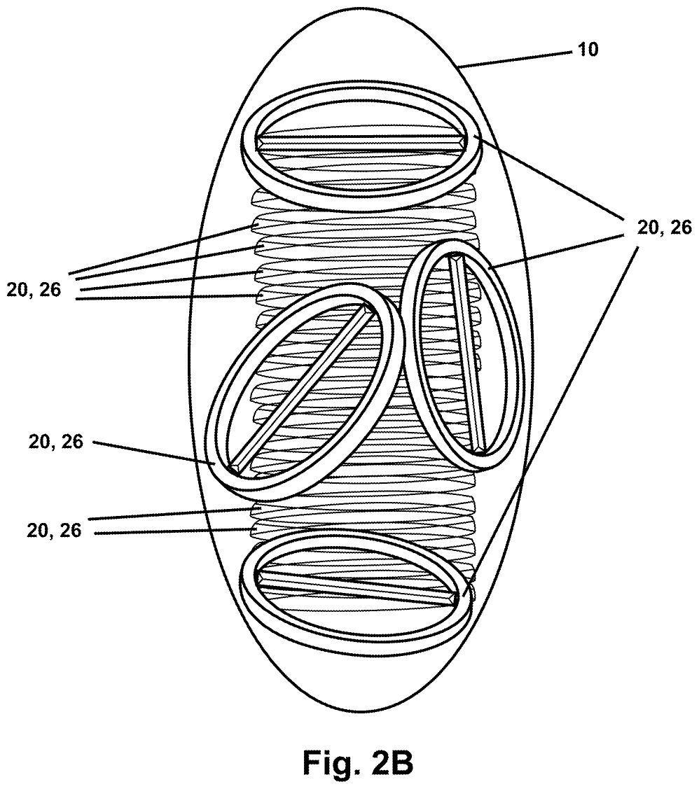

FIG. 2C shows an embodiment of an ingestible and dissolvable capsule filled with a plurality of radiopaque markers.

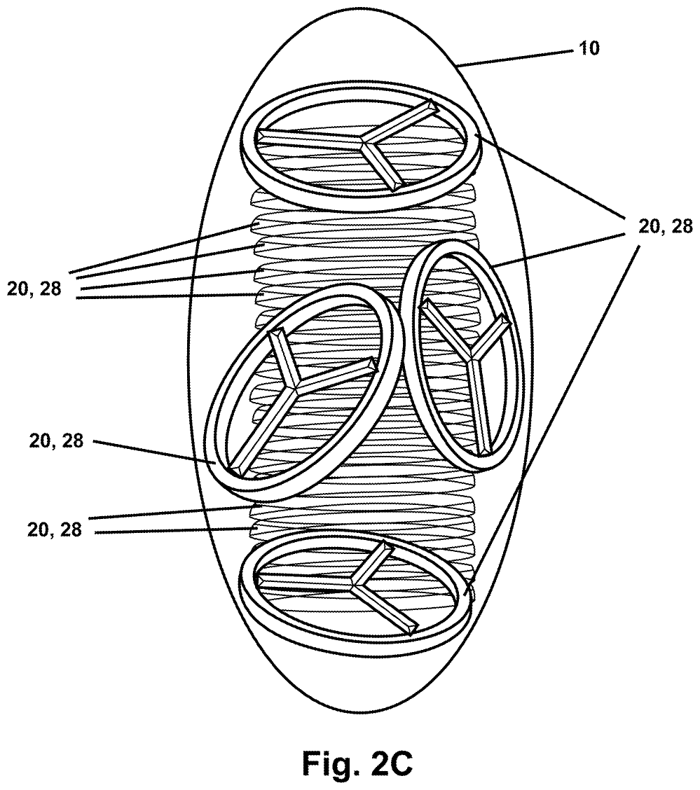

FIG. 3A shows an embodiment of a radiograph showing radiopaque markers within a pediatric patient's colon.

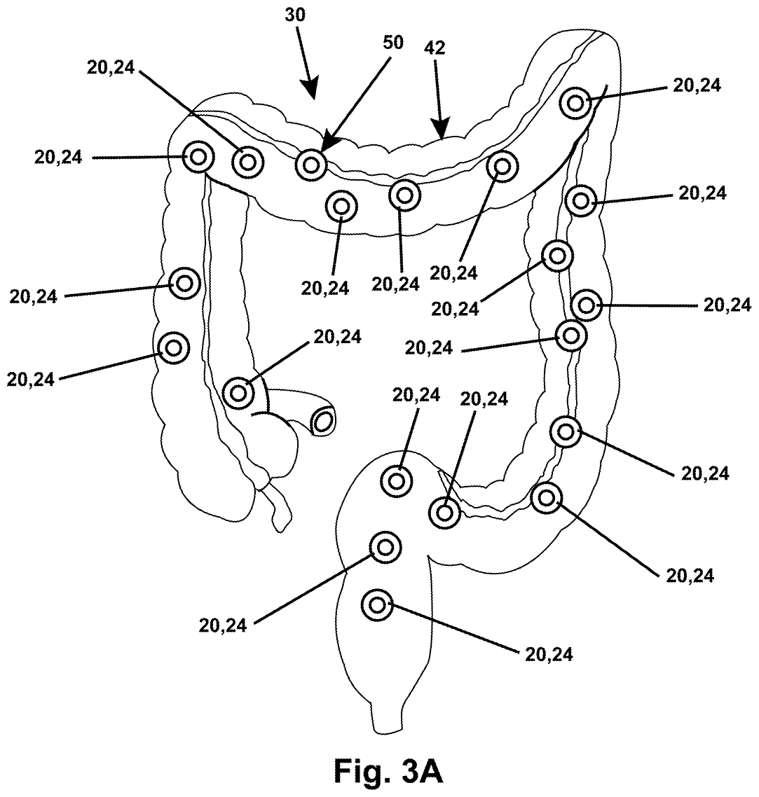

FIG. 3B shows an embodiment of a radiograph showing radiopaque markers within a pediatric patient's colon.

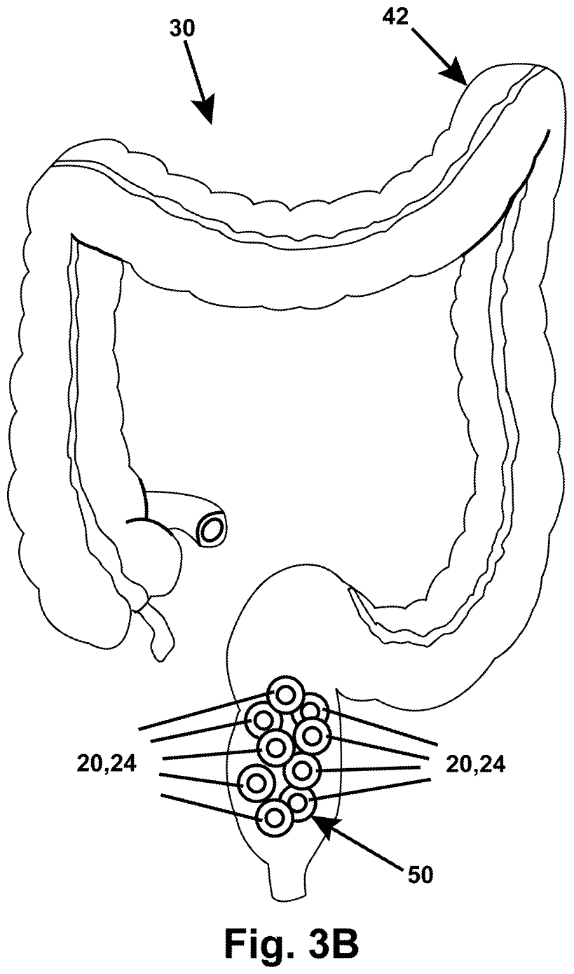

### DETAILED DESCRIPTION OF THE INVENTION

Embodiments directed to monitoring pediatric colonic motility are provided. Embodiments generally include administering an ingestible and dissolvable capsule 10 to a pediatric patient, wherein capsule 10 contains a plurality of radiopaque markers 20. The pediatric patient subsequently ingests capsule 10 that then dissolves within the pediatric patient—thereby releasing the plurality of radiopaque markers 20 into the pediatric patient's digestive tract, and more specifically, into the pediatric patient's colon 42. After radiopaque markers 20 are released from capsule 10 (and into the pediatric patient's digestive tract), a radiograph 30 is then performed on the pediatric patient's colon region in order to identify the location 50 of at least one of the plurality of radiopaque markers 20 within the pediatric patient's colon 42. Relative to the time of capsule ingestion, the time at which radiograph 30 is performed in combination with the location 50 of at least one of the radiopaque markers 20, can help to determine the pediatric patient's colonic-motility rate based upon a distance-per-time calculation. Any number of radiographs 30 can be performed on the pediatric patient's colon region as needed in order to monitor the location 50 and rate at which radiopaque markers 20 are moving through the pediatric patient's colon 42. X-ray radiographs 30 are useful types of radiographs 30 that can be used to monitor the location 50 of radiopaque markers 20 within the pediatric patient's colon 42.

As mentioned above, and in order to monitor pediatric colonic motility, these pediatric embodiments use an ingestible and dissolvable capsule 10 filled with a plurality of radiopaque markers 20. These types of ingestible and dissolvable capsules 10 that are filled with radiopaque markers 20 are known to be used in adult colonic-motility applications but not in pediatric-patient applications because of a previous lack of U.S. governmental approval for use in the pediatric setting. As a non-limiting example of a useful and commercially available product that can be used in these pediatric colonic-motility embodiments, Konsyl Pharmaceuticals both previously and currently distributes the SITZMARKS® product for use in adult colonic-motility applications. The SITZMARKS® product is an ingestible and dissolvable capsule 10 having a plurality of radiopaque markers 20 therein; the SITZMARKS® product has been used previously in adult patients to monitor adult-colonic motility and can now be used in pediatric patients to monitor pediatric-colonic motility.

As an alternative to using the SITZMARKS® product, persons of ordinary skill in the art can manufacture useful capsules 10 that are filled with radiopaque markers 20 without having to exercise undue experimentation. In manufacturing an ingestible and dissolvable capsule 10, any known commercially available capsule 10 can be used to house radiopaque markers 20. Ingestible and dissolvable capsules 10 are commonly used to transport and deliver a wide variety of medicinal agents and pharmaceuticals to patients, and of those capsules 10 that are known, persons of ordinary skill in the art can select useful capsules 10 without exercising undue experimentation. As a non-limiting example, pharmaceutical grade vegetable gelatin can be used to manufacture a useful capsule 10, and pharmaceutical grade vegetable gelatin is known and commercially available. Pharmaceutical grade vegetable gelatin and capsules 10 made thereof are currently available from CAPSUGEL®.

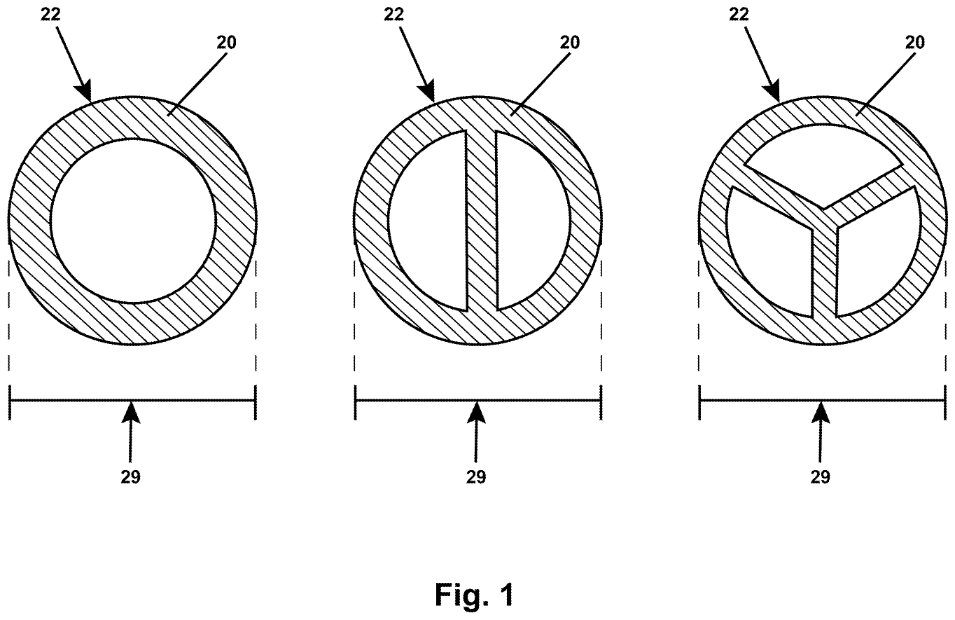

Useful shapes of radiopaque markers 20 are known, and any of them can be used in embodiments directed to monitoring pediatric colonic motility. As a non-limiting example, and as shown in the figures, useful perimeter shapes 22 of radiopaque markers 20 include closed-loop circular or substantially circular perimeter shapes. In embodiments having radiopaque markers 20 with perimeters that are closed-loop and substantially ring shaped, various internal configurations within the substantially ring-shaped perimeter can also be used in order to distinguish some radiopaque markers 20 from others. As shown in the figures, internal configurations include no-bar, single-bar, and multiple-bar configurations. FIG. 1A shows a useful radiopaque-marker shape that is circular, ring-shaped, and having an open-space internal configuration; in an embodiment, this is a first kind of marker 24 having a perimeter shape that is substantially circular and having a first open-space internal configuration. FIG. 1B shows a useful radiopaque-marker shape that is circular, ring shaped, and having a single-internal-bar configuration; in an embodiment, this is a second kind of marker 26 having a perimeter shape that is substantially circular and having a second internal configuration. FIG. 1C shows a useful radiopaque-marker shape that is circular, ring shaped, and having a multiple-internal-bar configuration; in an embodiment, this is a third kind of marker 28 having a perimeter shape that is substantially circular and having a third internal configuration. Any of the radiopaque-marker shapes shown in FIGS. 1A, 1B, or 1C can be used alone or in combination within capsule 10. In some embodiments of the drawings, the radiopaque markers 20 are intentionally labeled with two numerical identifiers; in those embodiments having two numerical identifiers, the numerical identifier “20” indicates that it is a radiopaque marker, and the second numerical identifier, e.g., 24, 26, or 28, indicates the specific kind of radiopaque marker, e.g., i) a first kind of marker 24 having a perimeter shape that is substantially circular and having a first open-space internal configuration, ii) a second kind of marker 26 having a perimeter shape that is substantially circular, ring shaped, and having a single-internal-bar configuration, and iii) a third kind of marker 26 having a perimeter shape that is substantially circular, ring shaped, and having a multiple-internal-bar configuration.

Any known useful sizes of radiopaque markers 20 for adult colonic-motility rates can be used for monitoring pediatric colonic-motility rates. In an embodiment, a non-limiting list of useful sizes include circular or substantially circular perimeter shapes 22 having diameters 29 less than 0.2 inches, wherein the diameter 29 is from one perimeter outer edge to the opposite other. In other embodiments, useful sizes include circular or substantially circular perimeter shapes 22 having diameters 29 less than or equal to 0.180 inches, wherein the diameter 29 is from one perimeter outer edge to the opposite other. In other embodiments, useful sizes include circular or substantially circular perimeter shapes 22 having diameters 29 less than or equal to 0.177 inches, wherein the diameter 29 is from one perimeter outer edge to the opposite other.

In embodiments, useful sizes of radiopaque markers 20 are those that have a greatest measurable linear dimension that is less than or equal to 0.2 inches. In other embodiments, useful sizes of radiopaque markers 20 are those that have a greatest measurable linear dimension that is less than or equal to about 0.180 inches. In other embodiments, useful sizes of radiopaque markers 20 are those that have a greatest measurable linear dimension that is less than or equal to about 0.177 inches.

In embodiments, useful radiopaque markers 20 are substantially disk-shaped. As a non-limiting example, useful radiopaque markers 20 have a diameter 29 of less than 0.2 inches and a thickness of less than 0.1 inches. In other embodiments, useful radiopaque markers 20 have a diameter 29 of less than 0.2 inches and a thickness of less than 0.08 inches. In other embodiments, useful radiopaque markers 20 have a diameter 29 of less than 0.2 inches and a thickness of less than 0.06 inches.

Any number of radiopaque markers 20 can be within ingestible and dissolvable capsule 10. In embodiments, less than 40 radiopaque markers 20 are within ingestible and dissolvable capsule 10. In embodiments, less than 35 radiopaque markers 20 are within ingestible and dissolvable capsule 10. In embodiments, less than 30 radiopaque markers 20 are within ingestible and dissolvable capsule 10. In embodiments, less than 25 radiopaque markers 20 are within ingestible and dissolvable capsule 10. In embodiments, less than 20 radiopaque markers 20 are within ingestible and dissolvable capsule 10. In embodiments, less than 15 radiopaque markers 20 are within ingestible and dissolvable capsule 10.

In embodiments, at least 5 radiopaque markers 20 are within ingestible and dissolvable capsule 10. In embodiments, at least 10 radiopaque markers 20 are within ingestible and dissolvable capsule 10. In embodiments, at least 15 radiopaque markers 20 are within ingestible and dissolvable capsule 10. In embodiments, at least 20 radiopaque markers 20 are within ingestible and dissolvable capsule 10. In embodiments, 24 radiopaque markers 20 are within ingestible and dissolvable capsule 10.

In an embodiment, all radiopaque markers 20 within ingestible and dissolvable capsule 10 have substantially the same shape and internal configuration. In an embodiment, at least two radiopaque markers 20 within ingestible and dissolvable capsule 10 have different shapes, e.g., different internal configurations. In another embodiment, at least three radiopaque markers 20 within ingestible and dissolvable capsule 10 have different shapes, e.g., different internal configurations. In still another embodiment, at least four radiopaque markers 20 within ingestible and dissolvable capsule 10 have different shapes, e.g., different internal configurations.

Useful radiopaque markers 20 can be manufactured using any known composition that is radiopaque, e.g., visible on an X-ray radiograph 30. In embodiments, radiopaque markers 20 are manufactured using a composition that includes a polymer and at least one radiopaque filler or radiopaque component. Barium sulfate is an example of a known filler or component that is radiopaque. As a non-limiting example, radiopaque marker 20 could be manufactured using a polyvinylchloride polymer filled with barium sulfate and further including known stabilizer(s) and known plasticizer(s). Useful and known stabilizers include calcium-zinc organic soap that is commercially available; useful and known platicizer stabilizers include epoxidized soybean oil; and useful and known plasticizers include di-2-ethyl hexylphthalate. Useful amounts of the polymer, filler, stabilizers, and plasticizers can be determined by someone of ordinary skill in the art without having to exercise undue experimentation.

Embodiments are directed to monitoring pediatric colonic motility in pediatric patients that are between 2 and 17 years old. Some embodiments are directed to monitoring pediatric colonic motility in pediatric patients that are between 2 and 14 years old. Some embodiments are directed to monitoring pediatric colonic motility in pediatric patients that are between 2 and 8 years old.

In embodiments, one or more radiographs 30 are performed on a pediatric patient after the pediatric patient has ingested ingestible and dissolvable capsule 10 filled with a plurality of radiopaque markers 20; the purpose of performing a radiograph 30 is to identify the location 50 of one or more of the plurality of radiopaque markers 20 in the pediatric patient's colon 42 or digestive tract. A radiograph 30 can be performed at any time after a pediatric patient has ingested ingestible and dissolvable capsule 10 filled with a plurality of radiopaque markers 20. In an embodiment, a radiograph 30 is performed one day after ingestion. In an embodiment, a radiograph 30 is performed two days after ingestion. In an embodiment, a radiograph 30 is performed three days after ingestion. In an embodiment, a radiograph 30 is performed four days after ingestion. In an embodiment, a radiograph 30 is performed five days after ingestion. In an embodiment, a radiograph 30 is performed six days after ingestion. In an embodiment, a radiograph 30 is performed seven days after ingestion. In an embodiment, a radiograph 30 is performed less than six days after the pediatric patient ingests ingestible capsule 10. In an embodiment, a radiograph 30 is performed less than seven days after the pediatric patient ingests ingestible capsule 10.

Any known radiograph 30 can be performed on the pediatric patient. As a non-limiting example, an X-ray radiograph 30 can be used to identify the location 50 of the plurality of radiopaque markers 20 in the pediatric patient's colon 42 or digestive tract.

In an embodiment, colon transit time (CTT) is calculated by performing a radiograph 30 and then determining the distance traveled by one or more radiopaque markers 20 per time; this distance per time calculation will give a rate of radiopaque movement within the pediatric patient's colon 42. In another embodiment, the number of radiopaque markers 20 within the patient after a period of time can be used to calculate colon transit time; as a non-limiting example, the number of radiopaque markers 20 within a patient (in combination with the respective marker location(s) 50 within the pediatric patient) three to seven days after ingestion can be used to determine colon transit time.

What is claimed is:

1. A method for monitoring pediatric colonic motility comprising:
administering an ingestible and dissolvable capsule to a pediatric patient, wherein the capsule contains a plurality of radiopaque markers; and
performing a radiograph on the pediatric patient to identify the location of at least one of the plurality of radiopaque markers within the pediatric patient's colon.

2. The method of claim 1, wherein the radiograph is performed less than six days after the pediatric patient ingests the ingestible capsule.

3. The method of claim 1, wherein the radiograph is performed three days after the pediatric patient ingests the ingestible capsule.

4. The method of claim 1, wherein the ingestible capsule contains at least 10 radiopaque markers.

5. The method of claim 1, wherein the ingestible capsule contains at least 15 radiopaque markers.

6. The method of claim 1, wherein the ingestible capsule contains at least 20 radiopaque markers.

7. The method of claim 1, wherein the ingestible capsule contains 24 radiopaque markers.

8. The method of claim 1, wherein all of the radiopaque markers have a perimeter shape that is substantially circular.

9. The method of claim 1, wherein there are at least two different shapes of radiopaque markers:
a first kind of marker having a perimeter shape that is substantially circular and having a first internal configuration, and a second kind of marker having a perimeter shape that is substantially circular and having a second internal configuration.

10. The method of claim 1, wherein there are at least three different shapes of radiopaque markers: a first kind of marker having a perimeter shape that is substantially circular and having a first internal configuration; a second kind of marker having a perimeter shape that is substantially circular and having a second internal configuration, and a third kind of marker having a perimeter shape that is substantially circular and having a third internal configuration.

11. The method of claim 1, wherein the pediatric patient has an age ranging from 2 to 17 years old.

12. The method of claim 1, wherein the pediatric patient has an age ranging from 2 to 14 years old.

13. The method of claim 1, wherein the pediatric patient has an age ranging from 2 to 8 years old.

14. The method of claim 1, wherein all of the radiopaque markers have a perimeter shape that is substantially circular and a diameter that is less than 0.180 inches.

15. The method of claim 1, wherein the radiograph is an X-ray radiograph.

16. A method for monitoring pediatric colonic motility comprising:
administering an ingestible and dissolvable capsule to a pediatric patient having an age ranging from 2 to 17 years old, wherein the capsule contains a plurality of radiopaque markers; and
performing a radiograph on the pediatric patient to identify the location of at least one of the plurality of radiopaque markers within the pediatric patient's colon;
wherein the radiograph is performed less than six days after the pediatric patient ingests the ingestible capsule,
wherein the ingestible capsule contains at least 5 radiopaque markers,
wherein there are at least three different shapes of radiopaque markers: a first kind of marker having a perimeter shape that is substantially circular and having a first internal configuration; a second kind of marker having a perimeter shape that is substantially circular and having a second internal configuration, and a third kind of marker having a perimeter shape that is substantially circular and having a third internal configuration, and
wherein all of the radiopaque markers have a perimeter shape that is substantially circular and a diameter that is less than 0.180 inches.

17. A method for monitoring pediatric colonic motility comprising:
administering an ingestible and dissolvable capsule to a pediatric patient having an age ranging from 2 to 17 years old, wherein the capsule contains 24 radiopaque markers; and
performing a radiograph on the pediatric patient to identify the location of at least one of the plurality of radiopaque markers within the pediatric patient's colon;
wherein the radiograph is performed less than six days after the pediatric patient ingests the ingestible capsule,
wherein the ingestible capsule contains at least 5 radiopaque markers,
wherein there are at least three different shapes of radiopaque markers: a first kind of marker having a perimeter shape that is substantially circular and having a first internal configuration; a second kind of marker having a perimeter shape that is substantially circular and having a second internal configuration, and a third kind of marker having a perimeter shape that is substantially circular and having a third internal configuration, and
wherein all of the radiopaque markers have a perimeter shape that is substantially circular and a diameter that is less than 0.180 inches.
